(19) United States
(12) Patent Application Publication
(10) Pub. No.: US 2005/0143932 A1
(43) Pub. Date: Jun. 30, 2005

(54) Meal planning tool

(76) Inventor: Hamid A. Emminger, Mannheim (DE)

Correspondence Address: CASTELLANO MALM FERRARIO & BUCK PLLC, 2121 K STREET, NW, SUITE 800, WASHINGTON, DC, 20037, US

(21) Appl. No.: 11/020,267

(22) Filed: Dec. 27, 2004

Related U.S. Application Data
(60) Provisional application No. 60/533,984, filed on Dec. 31, 2003.

(51) Int. Cl. G06F 19/00; G01N 33/48; G01N 33/50; G06F 17/00

(52) U.S. Cl. 702/19; 707/102

(57) ABSTRACT

The present invention provides methods, systems, and products which overcome the problems of achieving and maintaining an adequate weight loss in the context of an existing obesity. The present invention makes dietary meal planning easier by eliminating the need to calculate the recommended daily calorie intake.

### CROSS-REFERENCE TO RELATED APPLICATION

The present application claims priority of U.S. provisional application No. 60/533,984 the whole contents of which is hereby explicitly incorporated by reference.

### BACKGROUND OF THE INVENTION

1. Field of the Invention

The present invention relates to a method, system, and product which overcome the problems of achieving and maintaining an adequate weight loss in the context of an existing obesity.

### BACKGROUND OF THE INVENTION

Obesity is a common and rapidly growing condition in the United States. According to the Centers for Disease Control and Prevention (CDC) and Behavioral Risk Factor Surveillance System (BRFSS) in the 1999 National Health and Nutrition Examination Survey (NHANES) 63% of men and 55% of women were overweight (defined by a Body Mass Index between 25 to 29.9) More recent (1999) data from NHANES IV found that obesity (defined by a Body Mass Index>30) rates among adult Americans increased from 15% in 1980 to 27% in 1999. Recent reports suggest an emerging epidemic of obesity which is directly related to the increasing dietary fat and calories. The annual economic costs of obesity in the United States are estimated to be more than 70 billion dollars.

The increasing prevalence of overweight and obesity and the corresponding incidence of obesity-related diseases underscore the need for preventing and treating the condition of obesity rather than focusing solely on its associated health risks. According to scientific literature, obesity is closely associated with the increasing risk of a number of complications that can occur alone or concomitantly. These include hypertension, dyslipidemia, cardiovascular disease, stroke, type 2 diabetes, gallbladder disease, respiratory dysfunction, gout and osteoarthritis. And there are still others—certain cancers, for example.

Fortunately, even modest weight loss reduces health risks. A sustained “weight loss as little as 5% to 10% has been shown to improve disorders associated with obesity”. Even relatively modest decreases in weight in the obese can result in substantial health benefits. Weight loss of 10% or less is often associated with marked clinical improvement. In fact, NAASO, NIH and WHO recommend weight loss also for those obesity-related disorders, like diabetes, hypertension, dyslipidemia, heart attack and stroke.

Although attempts to lose weight are common in the United States (36.2% of the population in the 1998 BRFSS were trying to lose weight), the prevalence of overweight and obesity continues to increase. An additional 55.6% of respondents in the 1998 BRFSS indicated they were trying to maintain their current bodyweight. One third of the respondents indicated they were consuming fewer calories and fat.

Beside the medical and psychological treatment healthy lifestyle habits and in particular a regular meal plan and a well balanced and reduced amount of calories per day are recommended as the cornerstone of obesity treatment.

The patient is often advised to follow a meal plan which limits the daily intake of calories to a certain level according to the existing or targeted weight.

However, other studies indicate that most persons attempting to lose weight are not using sound dietary principles. Achieving and retaining an appropriate weight remains a challenge to the individuals. Especially the calculation of the recommended calorie intake within the day to day meal plan seems to be too complicated.

Unfortunately, being diagnosed with a disease may not be enough or a motivating consequence for most patients. Therefore the modification of the lifestyle towards a low calorie diet has proven to be a major issue especially for the type II diabetics. Upon experts opinion the reasons include a usually advanced age of the patients and a lack of understanding for the disease and its consequences. But the main reason why patient's motivation for a diet usually remains limited is the fact that the existing tools and guidelines to achieve and maintain an adequate weight loss are often too complicated leading to a reduced motivation and willingness of the patients.

Especially calorie calculations turn out to be a major obstacle for elderly persons who are often not capable or willing to spend a considerable time measuring and adding calorie levels of different ingredients of a planned meal.

The need for intuitive and comprehensive tools for meal planning remains highly evident.

### SUMMARY OF THE INVENTION

The present invention provides methods, systems, and products which overcome the problems by achieving and maintaining an adequate weight loss in the context of an existing obesity. The present invention simplifies dietary meal planning by eliminating the need to calculate the recommended daily calories.

The present invention comprises several partitions or arrangements for daily meals (e.g. 3 main meals and 1 snack). Every meal partitions contain a number of recipe cards or suggestions. Each of these cards offers on its part a pre-calculated meal option.

All of the dishes within a meal partitions are coordinated with one another, because every meal option within a meal partitions contains approximately the same amount of calories, e.g. 300 calories. So one can choose what he/she fancies from a large number of options.

The addition of one card from every one of the several partitions leads to a pre-calculated and recommended amount of daily calorie intake, e.g. 1600 calories. Hence by selecting a card for each meal from the box one automatically achieves a preset daily calorie intake.

### BRIEF DESCRIPTION OF THE DRAWINGS

FIG. 1 shows a schematic perspective view of an embodiment according to the invention, which is in form of a box (“DietBox”) with different partitions containing pre-calculated recipe cards.

### DETAILED DESCRIPTION OF THE INVENTION

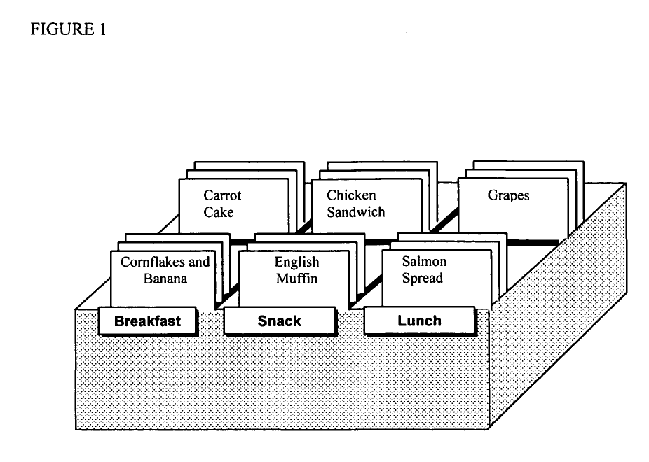

The meal card set “DietBox” simplifies dietary meal planning by eliminating the need to calculate the recommended daily calories. It contains several partitions for daily meals (e.g. 3 main meals and 1 snack). Every meal partitions contain a number of recipe cards. Each of these cards offers on its part a pre-calculated meal option (see FIG. 1).

All of the dishes within a meal partitions are coordinated with one another in the way that every meal option within a meal partitions contains approximately the same amount of calories, e.g. 300 calories, so one can choose what he/she fancies from a large number of options. The addition of one card from every one of the several partitions leads to a pre-calculated and recommended amount of daily calorie intake, e.g., 1600 calories. Of course, the total daily caloric intake can be less than or greater than 1600 calories.

Hence by selecting a card for each meal from the box one automatically achieves a preset daily calorie intake.

The “DietBox” arrangement can not only be applied to a box concept for different amounts of daily calories. It can also be used for cooking books combining different pre-calculated recipes or electronic applications (e.g. CD-ROM, Internet, databanks etc.).

The following instruction demonstrates a preferred embodiment of the invention for a daily calorie intake of 1400 calories.

During the day the user selects each a card for breakfast, lunch and evening meal from the recipe set, and also choose two snacks. All of the recipes for a given meal provide roughly the same number of calories.
Breakfast: about 300 kcal
Lunch: about 350 to 380 kcal
2 snacks: about 200 kcal each
Evening meal: about 350 kcal

Hence, at the end of the day the user will “automatically” have arrived at an approximate amount of 1400 calories.

1. A method comprising the systematic arrangement of a plurality of meal recipes according to the meals' energy levels, wherein every recipe comprises preparation instructions and a statement on the energy amount of said meal.

2. The method of claim 1, wherein said systematic arrangement divides said plurality of meal recipes in about 2 to 10 groups.

3. The method of claim 2, wherein said systematic arrangement divides said plurality of meal recipes into about 3 to 8 groups.

4. The method of claim 3, wherein said systematic arrangement divides said plurality of meal recipes into 6 groups.

5. The method of claim 4, wherein said meals within the same meal recipe group contain approximately equal levels of energy, within about ±20%.

6. The method of claim 5, wherein said energy levels of said meals are within about ±10%.

7. A method of weight reduction in obese humans, comprising the method of claim 1.

8. A method of weight reduction in diabetic humans, comprising the method of claim 1.

9. A system comprising an arrangement of a plurality of meal recipe cards, wherein each of said recipe cards comprises an illustration of the fully prepared meal, preparation instructions and statements on the energy level of said meal.

10. The system of claim 9, wherein said system comprises a plurality of partitions, wherein each of said partitions comprises meal recipes, wherein the energy levels of said meals within the same meal recipe partition are approximately equal, within about ±20%.

11. The system of claim 10, wherein said energy levels of said meals are within about ±10%.
